(19) United States
(12) Patent Application Publication
(10) Pub. No.: US 2022/0331187 A1
(43) Pub. Date: Oct. 20, 2022

(54) DYNAMIC MOTION BOOT AND RELATED METHODS FOR BLOOD CLOT PREVENTION

(71) Applicant: DHD Partners, LLC, Frisco, TX (US)

(72) Inventors: Steven DeLeon, Austin, TX (US); Bart Dalton, Frisco, TX (US); William Head, Frisco, TX (US)

(21) Appl. No.: 17/639,830

(22) PCT Filed: Sep. 9, 2020
(86) PCT No.: PCT/US2020/049829

§ 371 Date: Mar. 2, 2022

Related U.S. Application Data
(60) Provisional application No. 62/898,228, filed on Sep. 10, 2019.

(51) Int. Cl. A61H 1/02

(52) U.S. Cl. CPC A61H 1/0266; A61H 2201/1676; A61H 2201/1642; A61H 2201/018; A61H 2201/1215; A61H 2201/5025; A61H 2201/165; A61H 2209/00

(57) ABSTRACT

A medical apparatus has a base portion shaped and configured to engage a proximal portion of a user's limb, a mobile portion shaped and configured to engage a distal portion of the user's limb, and a motor assembly configured to selectively rotate the mobile portion relative to the base portion. Related methods are disclosed herein.

### CROSS-REFERENCE TO RELATED APPLICATIONS

This application claims priority to U.S. Provisional Application No. 62/898,228, filed Sep. 10, 2019, and entitled “DYNAMIC MOTION BOOT AND RELATED METHODS FOR BLOOD CLOT PREVENTION,” the entire disclosure of which is hereby incorporated by reference for all proper purposes.

### FIELD

This invention is related to medical devices. Specifically, but not intended to limit the invention, embodiments of the invention are related to devices for blood clot prevention.

### BACKGROUND

A number of injuries, surgeries, or other medical conditions cause patients to be or become prone to blood clots, particularly in the legs of patients with little mobility. Currently, the medical industry encourages patients to regularly flex their feet up or down to maintain circulation and reduce clots. For patients with limited flexibility, a tether may be provided. The tether may allow the patients to pull their feet in a desired direction. This solution, however, requires some mobility and hand strength on the part of the patients.

There thus remains a need for an improved ability to prevent blood clots for relatively immobile patients, and/or other new and innovative solutions.

### SUMMARY

An exemplary medical apparatus has a base portion shaped and configured to engage a proximal portion of a user's limb; a mobile portion shaped and configured to engage a distal portion of the user's limb; and a motor assembly configured to selectively rotate the mobile portion relative to the base portion.

An exemplary method includes providing a medical apparatus having a base portion shaped and configured to engage a proximal portion of a user's limb, a mobile portion shaped and configured to engage a distal portion of the user's limb, and a motor assembly configured to selectively rotate the mobile portion relative to the base portion. The method includes affixing the base portion to the proximal portion of the user's limb. The method includes affixing the mobile portion to a distal portion of the user's limb. The method includes, using a user interface, causing the motor assembly to rotate the mobile portion relative to the base portion.

### BRIEF DESCRIPTION ON THE DRAWINGS

FIG. 1 is a front perspective view of an exemplary medical apparatus;

FIG. 2 is another front perspective view of the apparatus in FIG. 1;

FIG. 3 is a rear perspective view of the apparatus in FIG. 1; and

FIG. 4 is a flowchart of an exemplary method.

### DETAILED DESCRIPTION

Before providing a detailed description of the device and related methods, it is noted that the Applicant has solved the problem of reducing blood clot formation for patients with limited mobility by providing a tether-free solution. Embodiments herein include a boot that passively dorsiflexes the foot and causes the calf muscles to contract, thereby promoting blood flow in the deep veins in the lower extremity. In some embodiments, the motion is in one plane, for example, from neutral to dorsiflexion. The device may be powered by a motor attached to the boot and may include battery power or means to access a power source. The motor may include a step motor that cycles and can be powered on and off by a direct or remote switch. In use, the boot may be placed on the patient immediately post-op in an off or deactivated state, to allow the patient to ambulate with the boot in place. In some embodiments, the device described herein is indicated for lower extremity surgery, venous status and patients confined to bed.

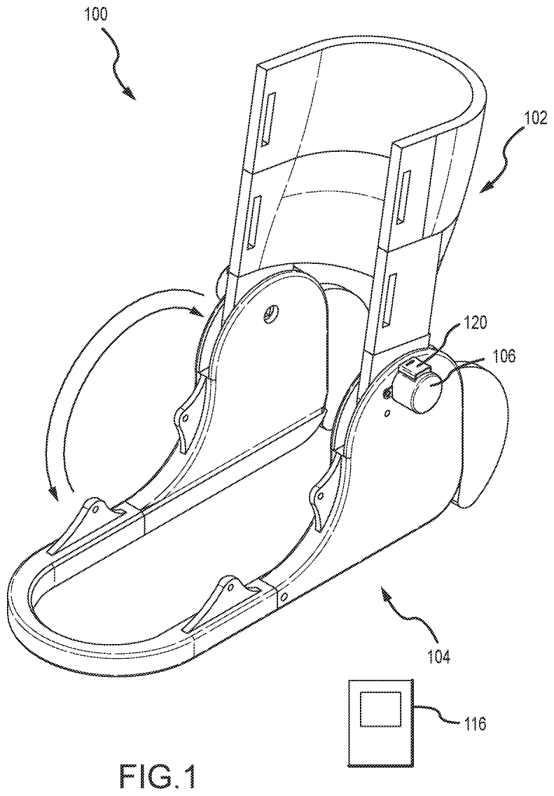

As illustrated in FIGS. 1-3, a medical apparatus 100, which may be interchangeably referenced herein as a boot, may be described herein as a dynamic motion boot. The boot 100 may be suitable for blood clot prevention in patients with limited mobility or having undergone recent surgical procedures.

The boot 100 may include a first portion 102, which may be referenced herein interchangeably as a base portion, suitable for affixing the boot to, for example, the lower portion of a patient's leg and/or the heel portion of a patient's foot. The base portion 102 may be attachable or may engage a proximal portion of a user's limb.

The boot 100 may include a second portion 104 suitable for affixing to, for example, a patient's foot. The sole of a patient's foot may rest on the second section. The second portion 104 may include one or more attachment mechanism 118 for removably attaching the patient's foot to the second portion 104. The attachment mechanism(s) 118 may include, without limitations, straps, buckles, hook and loop means, a platform-lever engagement, or any other suitable attachment means. The second portion 104 may be referenced herein interchangeably as a mobile portion. The mobile portion 104 may be configured to engage or be attached to a distal portion of the user's limb.

For the purpose of this document, the proximal portion of the user's limb may be the lower leg, and the distal portion of the user's limb may be the foot. The proximal portion of the user's limb may be the sole of the user's foot, and the distal portion of the user's limb may be the sole of the user's foot. The proximal portion of the user's limb may be the upper leg, and the distal portion may be the lower leg. The proximal portion may be the user's forearm, and the distal portion may be the user's hand. The proximal portion may be the user's upper arm, and the distal portion may be the user's hand.

Those skilled in the art will also recognize that the device is not limited for use on a human. For example, the device may be configured for use on primates or other animals.

The first and second portions 102, 104 may be movable relative to each other. The second portion 104 may be rotatable relative to the first portion 102. When applied to a user's foot, rotation of the second portion 104 may provide a dorsi flexion motion, wherein the foot is flexed upward toward the leg. Rotation of the second portion 104 may provide a plantar flexion motion, wherein the foot is flexed downward toward the sole.

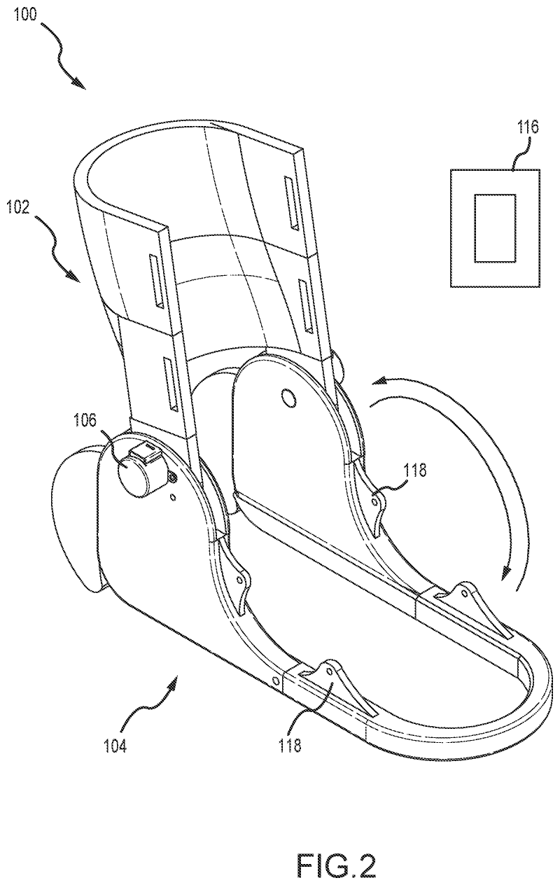

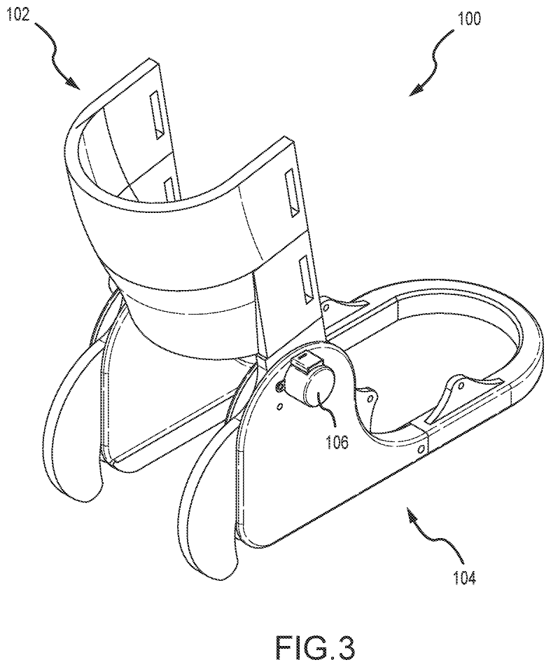

In some embodiments, and as seen most clearly in FIG. 3, a motor assembly 106 may be provided. The motor 106 may include a drive and/or gearing for providing a rotation of the second portion 104 relative to the first portion 102. As shown by the arrows in FIG. 2, the motor assembly 106 may be configured to rotate the mobile portion 104 between a first position and a second position wherein a distal portion of the mobile portion 104 is closer to the base portion 102.

In some embodiments, a small motor and battery pack (not shown), such as, for example only, a lithium or 12V rechargeable battery pack, may be coupled to the first portion 102 and positioned to fit behind the patient's ankle. The motor assembly 106 and battery pack may be raised off the floor for easier walking. Those skilled in the art will recognize that a battery pack is not a requirement; instead, the device 100 may be configured to draw power from another power source, such as a wall outlet, a generator, or any other suitable power source.

A user interface 116, shown in FIG. 1, may be provided to enable a user to activate the device. The user interface 116 may include a remote control. The user interface 116 may include an activation switch 120 directly on the boot. The user interface may include an override switch 120 on the boot. Those skilled in the art will recognize that the user interface 116 and/or switch 120 must be positioned so as to provide access to the user, who may have limited mobility.

When the motor assembly 106 is activated, it may slowly rotate the second portion 104 relative to the first portion 102, for example, until an adjustable stop triggers a limit switch. The limit switch may deactivate the motor when reached to enable the wearer to lower the second portion 104 down to the starting position (plantarflexion). In some embodiments, a lifting cycle can restart when the wearer activates the motor assembly with a remote. This is a dorsi flexion motion.

In the alternative, or in addition, the motor assembly 106 may provide a plantar flexion motion in substantially the same manner, but with rotation and triggers being in the opposite direction.

In some embodiments, the motor assembly 106 may include a force limiter to prevent an over-application of force to a user whose foot or dexterity is severely damaged.

In some embodiments, in response to an input to the user interface 116, the motor assembly 106 may be configured to rotate the second portion 104 toward and away from the first portion 102 for a preselected number of cycles. The motor assembly 106 may include a drive or gearing on both sides of the boot, or the motor assembly 106 may include a driver on a first side and an alignment mechanism, guide, or gear, on an opposing side.

Although not shown, those skilled in the art will recognize that the boot 100 may include a variety of coverings for aesthetic and/or sanitation purposes. For example, the coverings may be replaceable, for sanitary transfer between multiple patients, and/or the coverings may provide comfort and aesthetic appeal for a single wearer.

Hook and loop fasteners and/or any other fasteners (not shown) may be provided to allow a user to affix the boot 100 to the wearer.

In some embodiments, the boot 100 may be specifically shaped to fit a user's right foot only, or a user's left foot only. In some embodiments, the boot 100 may be universal.

Those skilled in the art will recognize that the boot 100 may include a variety of programmable functions, such as, for example only, a repetitive lift and/or lower cycle, a time-out limit, a force limit (for example, where the patient's foot is unable to rotate passively beyond a point prior to reaching the pre-set limit), and so forth.

Those skilled in the art will further recognize that, although not shown, any manner of providing comfort at the interface between the user and the device 100 is contemplated herein, including, but not limited to, a velfoam garment interface between the user and the device 100, a shoe, a specialized boot form, etc.

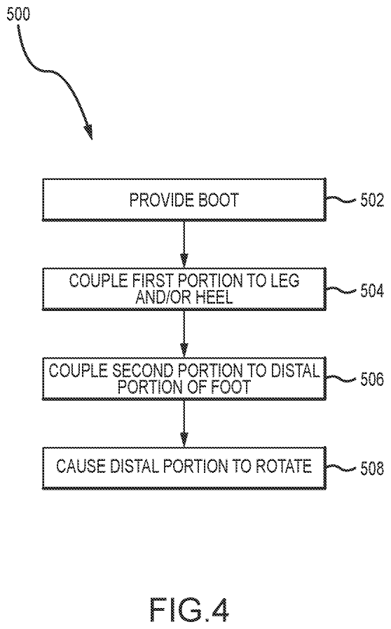

Turning now to FIG. 4, a method 500, such as a method of preventing blood clots in human patients is described. The method 500 may include providing 502 a medical apparatus, which may be referenced herein as a boot. The boot may be substantially as described herein with reference to the boot 100.

Providing 502 a medical apparatus may include providing a medical apparatus having a base portion shaped and configured to engage a proximal portion of a user's limb, a mobile portion shaped and configured to engage a distal portion of the user's limb, and a motor assembly configured to selectively rotate the mobile portion relative to the base portion.

The method 500 may include coupling 504 the first portion of the boot to at least one of the user's leg or the user's heel. Coupling 504 may also be described as affixing the base portion to the proximal portion of the user's limb.

The method 500 may include coupling 506 the second portion of the boot to a distal portion of the user's foot. Coupling 506 may also be described as affixing the mobile portion to a distal portion of the user's limb.

The method 500 may include, using a user interface, causing 508 the motor assembly to rotate the mobile portion relative to the base portion. Causing 508 may include causing a distal portion of the medical apparatus to rotate relative to a proximal portion of the apparatus.

Each of the various elements disclosed herein may be achieved in a variety of manners. This disclosure should be understood to encompass each such variation, be it a variation of an embodiment of any apparatus embodiment, a method or process embodiment, or even merely a variation of any element of these. Particularly, it should be understood that the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which this invention is entitled.

As but one example, it should be understood that all action may be expressed as a means for taking that action or as an element which causes that action. Similarly, each physical element disclosed should be understood to encompass a disclosure of the action which that physical element facilitates. Regarding this last aspect, the disclosure of a “fastener” should be understood to encompass disclosure of the act of “fastening”—whether explicitly discussed or not—and, conversely, were there only disclosure of the act of “fastening”, such a disclosure should be understood to encompass disclosure of a “fastening mechanism”. Such changes and alternative terms are to be understood to be explicitly included in the description.

Moreover, the claims shall be construed such that a claim that recites “at least one of A, B, or C” shall read on a device that requires “A” only. The claim shall also read on a device that requires “B” only. The claim shall also read on a device that requires “C” only.

Similarly, the claim shall also read on a device that requires “A+B”. The claim shall also read on a device that requires “A+B+C”, and so forth.

The claims shall also be construed such that any relational language (e.g. perpendicular, straight, parallel, flat, etc.) is understood to include the recitation “within a reasonable manufacturing tolerance at the time the device is manufactured or at the time of the invention, whichever manufacturing tolerance is greater”.

Those skilled in the art can readily recognize that numerous variations and substitutions may be made in the invention, its use and its configuration to achieve substantially the same results as achieved by the embodiments described herein.

Accordingly, there is no intention to limit the invention to the disclosed exemplary forms. Many variations, modifications and alternative constructions fall within the scope and spirit of the invention as expressed in the claims.

What is claimed is:

1. A medical apparatus, comprising:
a base portion shaped and configured to engage a proximal portion of a user's limb;
a mobile portion shaped and configured to engage a distal portion of the user's limb; and
a motor assembly configured to selectively rotate the mobile portion relative to the base portion.

2. The medical apparatus of claim 1, wherein:
the motor assembly is configured to rotate the mobile portion between a first position and a second position wherein a distal portion of the mobile portion is closer to the base portion.

3. The medical apparatus of claim 1, wherein:
the motor assembly further comprises a force limiter.

4. The medical apparatus of claim 1, further comprising:
a user interface configured to control the motor assembly in response to a user input.

5. The medical apparatus of claim 1, further comprising:
a non-slip tread on at least one of the base portion or the mobile portion.

6. The medical apparatus of claim 1, wherein:
the mobile portion is selectively detachable from the base portion.

7. A method, comprising:
providing a medical apparatus having a base portion shaped and configured to engage a proximal portion of a user's limb, a mobile portion shaped and configured to engage a distal portion of the user's limb, and a motor assembly configured to selectively rotate the mobile portion relative to the base portion;
affixing the base portion to the proximal portion of the user's limb;
affixing the mobile portion to a distal portion of the user's limb; and
using a user interface, causing the motor assembly to rotate the mobile portion relative to the base portion.

8. The method of claim 7, further comprising:
the motor assembly is configured to rotate the mobile portion between a first position and a second position wherein a distal portion of the mobile portion is closer to the base portion.

9. The method of claim 7, wherein:
the motor assembly further comprises a force limiter.

10. The method of claim 7, wherein:
the medical apparatus has a non-slip tread on at least one of the base portion or the mobile portion.

11. The method of claim 7, wherein:
the mobile portion is selectively detachable from the base portion.
